(19) United States
(12) Patent Application Publication
(10) Pub. No.: US 2011/0033917 A1
(43) Pub. Date: Feb. 10, 2011

(54) SENSOR WITH LONG-TERM STABILITY FOR BIOPROCESSES

(75) Inventors: Thilo Trapp, Waldheim (DE); Michael Hanko, Dresden (DE); Ânnett Planitzer, Lichtenau (DE)

Correspondence Address: BACON & THOMAS, PLLC, 625 SLATERS LANE, FOURTH FLOOR, ALEXANDRIA, VA, 22314-1176, US

(73) Assignee: ENDRESS + HAUSER CONDUCTA GESELLSCHAFT FÜR MESS- UND REGELTECHNIK MBH + CO. KG, Gerlingen (DE)

(21) Appl. No.: 12/734,112

(22) PCT Filed: Oct. 9, 2008
(86) PCT No.: PCT/EP2008/063543

§ 371 Date: Oct. 15, 2010

(30) Foreign Application Priority Data
Oct. 11, 2007 (DE) 102007049013.7

(51) Int. Cl. C12M 1/34

(52) U.S. Cl. 435/288.7; 435/287.1

(57) ABSTRACT

A sensor for registering a measured variable of a medium, especially in a bioprocess. The sensor includes a sensor body, wherein at least one surface section of the sensor body can be supplied with the medium, and wherein a condition of this surface section affects the measured value. The sensor is characterized by the fact that the surface section contains a substance with biocidal properties.

The present invention relates to a sensor (especially a pH sensor or another potentiometric sensor, or an optical sensor, such as a photometric sensor, a turbidity sensor, or a spectrometric sensor) for monitoring a physical or chemical parameter.

Such sensors are applied, among other things, for monitoring so called bioprocesses, in the case of which often especially large requirements as regards stable process conditions and purity are placed.

Standing in the way of this is that some of these sensors, especially pH sensors, have a transducer with a variable transfer function and, insofar, must, from time to time, be calibrated, subjected to maintenance or replaced as a function of the degree of change of the transfer function. Such calibrations, or maintenance, mean interventions in the bioprocess, in order to withdraw the sensor, or to install a new sensor. Similar calibrations or maintenance can be required in the case of the named optical sensors, when, for example, due to fouling, the transmission function of the optical path of the sensor changes.

It is, therefore, an object of the present invention to provide sensors of the named type with a lengthened service life, in order to be able to reduce the frequency of interventions in the process.

The object is achieved according to the invention by the sensor as defined in independent patent claim 1.

The sensor of the invention for registering a measured variable of a medium especially in a bioprocess includes a sensor body with at least one surface, wherein at least one surface section of the sensor body can be supplied with the medium, wherein the condition of this surface section affects the measured value,

characterized in that the surface section contains a substance with biocidal properties.

A bioprocess in the sense of the invention is, for example, a manufacturing, treating or cleaning process, in which microorganisms effect the conversion of media components.

A surface section contains a substance with biocidal properties in the sense of the invention, when it is suitable, in chemical, physical or biological ways, to destroy, to discourage, to make unharmful, to avoid damage from, or to combat in some other manner, harmful organisms.

In a currently preferred form of embodiment, the surface section contains a substance, which makes difficult, or completely suppresses, protein adsorption on the surface.

In a currently preferred, embodiment, the surface section contains a non-toxic substance.

Preferably, the substance is hydrophilic and, in given cases, water soluble, wherein the substance, further preferably, is anchored on the surface in such a manner in the region of the surface section, that it is not released under bioprocess conditions into the medium. The terminology ‘bioprocess conditions’ refers, on the one hand, to conditions as regards temperature, pressure, pH value and composition of the medium, including the microorganisms, under which a bioprocess usually transpires.

Preferably, the sensors are so formed, that the substance in the surface section survives, essentially undamaged, at least one cleaning, or sterilizing (CIP, i.e. cleaning in process, or SIP, i.e. sterilizing in process) in the installed condition, i.e. especially, without being dissolved away and without losing its biocidal properties. Additionally, preferably, the substance withstands a plurality of cleanings, or sterilizations. A first sterilizing of a sensor can be required, for example, after its installation in a process, before being charged with a medium to be processed. On the other hand, the installation can also be cleaned, or sterilized after the processing of a charge.

In a currently preferred embodiment, the substance of the invention comprises polyethylene glycol, which is immobilized in suitable manner on the surface of the body in the region of the surface section.

For immobilizing polyethylene glycol on a surface section comprising glass or a ceramic material, according to a further development of the invention, for example, silanes can be used.

In a further development of the invention, the polyethylene glycol has, for example, an average molecular weight of not less than 3000 Da, preferably not less than 4000 Da and further preferably not less than 4500 Da.

On the other hand, the polyethylene glycol has, according to the invention, for example, an average molecular weight of no greater than 7500 Da, preferably no greater than 6000 Da and further preferably no greater than 5500 Da.

In a currently preferred embodiment of the invention, the average molecular weight of the polyethylene glycol, amounts to, for instance, 5000 Da.

In the following, the functioning of polyethylene glycol (PEG) in the case of a sensor of the invention will now be explored in detail. The discussion of the mechanism of action is to be considered only for purposes of explanation of possible theory of how the invention works, not, however, for definition of the invention.

PEG is a hydrophilic, water soluble and non-toxic polymer, which exhibits very low interaction with proteins and permits an effective shielding of the surface. The increased resistance of PEG coated surfaces to protein adsorption can be attributed to different mechanisms. The most important are steric repulsion and hydration of the coated surface.

Steric repulsion occurs when a protein approaches a PEG surface and, in such case, provides a compressive pressure on the PEG molecules, which these meet with a correspondingly repelling counterpressure. Steric repulsion forces are an order of magnitude greater and have an essentially larger effect over a greater distance than possibly attractive electrostatic or van der Waals interactions.

Since PEG possesses the ability to arrange water molecules and hydrogen bonds around the ether oxygen, the PEGylized surface is strongly hydrated and proteins cannot deposit, or accrete, on the surface. Since many microorganisms utilize proteins for attaching to surfaces, their adhesion is reduced by the repelling forces. This was also already investigated and proven for bacteria cells and yeast cells.

The quantitative influence of chain length, degree of covering and the resulting structures (“brush” or “mushroom”) on the ability to repel protein is still the subject matter of current research. Fundamentally, however, both structures lead to that end.

In order to immobilize PEG on the surface section of a sensor of glass or ceramic, it is to be considered that the character of an SiO2 surface of glass is influenced strongly by the previous history of the material. Dependent on production process and conditions of storage, siloxane bonds are present, instead of silanol groups. In order to immobilize silanes on the surface as anchoring molecules for PEG, a pretreating of the glass surfaces is advantageous, in order to free the surface of impurities and to improve wettability. Additionally, the surface can be chemically activated, in order to create new silanol groups, which act as further bonding locations for a higher silane concentration. Freshly activated glass can have a density of silanol groups of up to 8 μmol/m².

After this so-called activating of the surface, there then follows the silanizing. In an embodiment of the invention, the silanizing can occur, for example, with an amino silane. For this, for example, commercially obtainable 3-aminopropyltriethoxysilane (APTES) can be used, wherein, for the silanizing reaction, for example, the organic solvent, toluol, can be applied.

The immobilizing of PEG on amino silanes can occur, according to the invention, for example, by reductive amination of methoxylated aldehyde terminated PEG (aldehyde PEG) or by nucleophilic substitution of PEG monomethyl ether mesylate, with mesylate as reactive, leaving group (mesylate PEG).

In an embodiment of the invention, the sensor comprises a pH sensor, in the form of a single-rod measuring chain or in the form of two separated, half cells, wherein the at least one surface section, which has the substance with the biocidal properties, can comprise a pH glass membrane and/or a diaphragm of the reference half-cell.

In another embodiment, the sensor is an optical sensor, such as a photometric sensor, a turbidity sensor, or a spectrometric sensor, wherein the at least one surface section, which has the substance with the biocidal properties, includes windows or other optical elements, by which the radiation of the sensor interacts with a measured medium, or by which the radiation of the sensor enters into, or escapes from, the measured medium.

The invention will now be explained using the example of a pH sensor of the invention illustrated in the drawing.

The figures of the drawing show as follows:

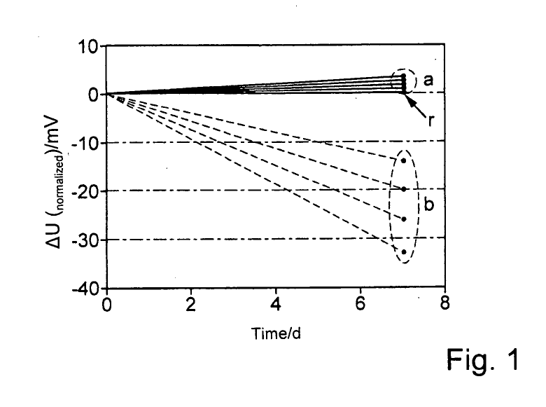

FIG. 1 a graph illustrating aging behavior of pH sensors in a fermenter;

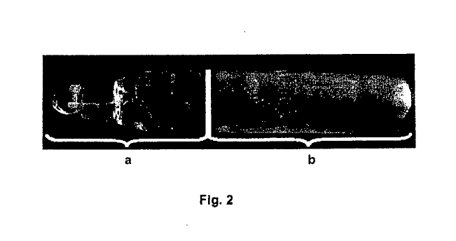

FIG. 2 a side view of a pH sensor of the invention in the form a single-rod measuring chain;

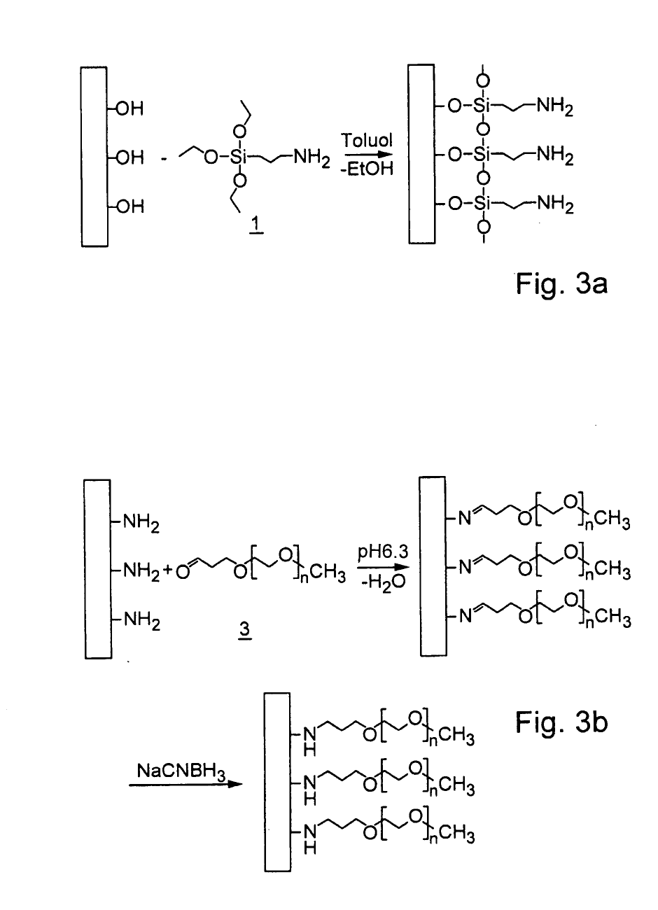

FIG. 3a the principle of silanizing the surface section of the sensor;

FIG. 3b the principle of immobilizing polyethylene glycol on the silanized surface section; and

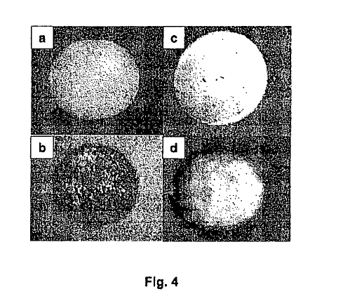

FIG. 4 detail photographs of pH sensor surfaces with the diaphragm of the reference half-cell for illustrating the effect of the invention.

The data in FIG. 1 show, as a function of time, the measurement signals of different pH sensors exposed to continuous yeast fermentation under aerobic conditions. Four single-rod, measuring chains of type CPS 71 of the assignee were coated with PEG according to the invention in a media contacting, surface section, which includes a pH glass membrane and a diaphragm of the reference half-cell. By way of illustration, FIG. 2 shows such a single-rod measuring chain, wherein the coating occurred in Section a, while Section b remained uncoated.

The four single-rod, measuring chains of the invention and four equally constructed, single-rod, measuring chains without coating were exposed for seven days continuously to yeast fermentation. The spread of the output signals of the sensors of the invention after seven days is shown by the circle labeled “a”, while the signals of the uncoated sensors at the same point in time are distributed by the ellipse labeled “b”.

A further, equally constructed sensor, whose signal is indicated with the arrow r, was only placed in the measured medium at the beginning of the experiment and again for a short time after a week, in order to obtain a reference value r. In the intervening times, it was stored without exposure to a measured medium.

The results show that the single-rod, measuring chains of the invention output in a fermentation process an essentially more stable measurement signal than the sensors of the state of the art.

Furthermore, it is significant and surprising that the coating of the pH glass membrane, or the diaphragm, with an amino silane and PEG clearly does not degrade the pH measurement. Both as regards the zero-point as well as also the slope of the pH potential, there are no mentionable deviations to detect relative to the reference electrode.

The reactions for implementing a sensor of the invention will now be explained on the basis of FIGS. 3a and 3b.

Immediately before the silanizing, the pH, single-rod, measuring chains (which henceforth will be referred to simply as the sensors) were cleaned 30 minutes in Piranha solution in an ultrasonic bath. Piranha solution is a 30% solution of 30% hydrogen peroxide solution in concentrated sulfuric acid. After the cleaning, the sensors were rinsed in deionized water and dried with compressed air.

The silanizing shown in FIG. 3a was performed in water free toluol. Under these conditions, there arises in the ideal case a uniform, monomolecular, polysiloxane layer. Used as silanizing reagent was 3-aminopropyltriethoxysilane (APTES) in the form of a 5% (v/v) solution in absolute toluol.

The cleaned sensors were silanized for half an hour. In this regard, 10 ml quantities of APTES solution were filled in 15 ml containers and into each vial was placed a sensor. A number of containers were placed in a glass beaker and the glass beaker moved by a shaking apparatus. In this way, a convective mixing of the medium was assured. After a silanizing time of 30 minutes, the sensors were rinsed with toluol and dried with compressed air. In the course of the investigations, the subsequent cross linking of the silane layer was found to be a decisive step of the amino silanizing. Samples, which were coated with PEG immediately after the silanizing, showed no coating success.

Therefore, the silanized sensors were stored at least 24 h in air at room temperature, in order to leave time for cross linking, and only then used for the PEG immobilizing.

A variant of the PEG coupling to the silanized glass surfaces is presented in FIG. 3b. It shows the immobilizing of methoxylated aldehyde terminated, PEG (aldehyde PEG), M-PEG-Ald, by reductive amination at the amino groups of the glass surface silanized with APTES.

The coupling of the reactive aldehyde groups to the amino groups of the silanized glass surface occurs under cloud point conditions via the forming of a Schiff base, which is reduced by the reducing agent, sodium cyanoborohydride, to a secondary amine. The reaction is performed in K₂SO₄ buffer solution (pH 6.3). This pH value assures that the reducing agent does not react with the carbonyl groups of the M-PEG-Ald.

The salt buffer solution, because of its poor solvent properties for PEG, reduces repelling, monomer-monomer interactions.

For the PEG immobilizing, an 11% potassium sulfate solution in di-sodium hydrogen-sodium dihydrogen-phosphate buffer was produced, which was set at a pH value of 6.3. Into this were added 1 mg/ml O-methyl-O′-(3-oxopropyl)-polyethylene glycol (aldehyde-PEG) and 3 mg/ml sodium cyanoborohydride.

Each of a series of 15 ml containers was filled with 10 ml of the PEG solution. The containers were placed in a glass beaker, which served as a water bath. The glass beaker was covered with aluminum foil and the PEG solutions therein heated on a heating plate to 60° C. After reaching this temperature, sensors were immersed, one in each container, and the water bath again “sealed” with foil. The applying of the PEG coating was performed for the duration of 6 hours at 60° C. Then, the samples were washed with deionized water, air dried and stored in a desiccator, before being used in the fermentation reactor.

Apart from the effect in the measurement behavior, the action of the invention can also be made visible directly on the sensor. FIG. 4 shows, in this connection, views of ceramic diaphragms of reference half-cells. Pictures a and b are of an uncoated sensor and pictures c and d of a sensor of the invention. Pictures a and c were taken before use in the bioprocess, while pictures b and d were taken following one week in the bioprocess.

The pictures show a marked growth on the untreated diaphragm, while such change on the diaphragm of the sensor of the invention cannot be detected. This correlates with the findings from FIG. 1, according to which the measuring signals of the sensors of the invention scarcely showed changes when used in yeast fermentation, while the measuring signals of the untreated sensors showed an aging dependent drift after a week in the medium.

1-10. (canceled)

11. A sensor for registering a measured variable of a medium, especially in a bioprocess, comprising:
a sensor body, wherein:
at least one surface section of said sensor body can be supplied with the medium;
a condition of said at least one surface section affects a measured value; and
in that said at least one surface section contains a substance with biocidal properties.

12. The sensor as claimed in claim 11, wherein:
said at least one surface section contains a substance, which makes difficult, or completely suppresses, protein adsorption on the surface.

13. The sensor as claimed in claim 11, wherein:
said substance with the biocidal properties is a non-toxic substance.

14. The sensor as claimed in one of claims 11, wherein:
said substance is hydrophilic and/or water soluble.

15. The sensor as claimed in claim 11, wherein:
said substance is anchored on the surface in such a manner in the region of said at least one surface section, that it is not released into the medium under bioprocess conditions.

16. The sensor as claimed in claim 11, wherein:
said substance in said at least one surface section survives at least one cleaning, or sterilizing, essentially undamaged, or without dissolving from said at least one surface section.

17. The sensor as claimed in claim 11, wherein:
said substance comprises polyethylene glycol, which is immobilized on the surface of the body in the region of said at least one surface section.

18. The sensor as claimed in claim 11, wherein:
said substance is anchored on a surface section of glass and/or ceramic by means of at least one silane species, especially 3-aminopropyltriethoxysilane (APTES).

19. The sensor as claimed in claim 11, wherein:
the sensor is a potentiometric sensor, especially a pH sensor, in the form of a single-rod measuring chain or in the form of two separated half cells; and
said at least one surface section, which contains the substance with the biocidal properties, can comprise a pH, glass membrane and/or a diaphragm of a reference half-cell.

20. The sensor as claimed in claim 11, wherein:
the sensor is an optical sensor; and
said at least one surface section, which contains the substance with the biocidal properties, comprises windows or other optical elements, through which radiation of the sensor interacts with a measured medium, or through whose media contacting surface radiation of the sensor is coupled in or out.
